(19) United States
(12) Patent Application Publication
(10) Pub. No.: US 2015/0086594 A1
(43) Pub. Date: Mar. 26, 2015

(54) ANTI-ALLERGIC MEDICAMENT

(71) Applicant: Mohan DEWAN, Pune, Maharasthra (IN)

(72) Inventor: Mohan Dewan, Maharashtra (IN)

(21) Appl. No.: 14/398,209

(22) PCT Filed: May 2, 2013
(86) PCT No.: PCT/IN2013/000291

§ 371 Date: Oct. 31, 2014

(30) Foreign Application Priority Data
May 2, 2012 (IN) 1363/MUM/2012

(51) Int. Cl. A61K 39/35; A61K 39/36

(52) U.S. Cl. CPC A61K 39/35; A61K 39/36; A61K 2039/545
USPC 424/275.1

(57) ABSTRACT

The present disclosure relates to a method for the treatment of allergic conditions. In said method the allergens to which an individual is susceptible is identified as per the allergy profile and accordingly a formulation having potentized dispersions of each of the identified allergens is administered to the individual for a predetermined period and at a predetermined potency. The present disclosure also relates to a formulation which is a mixture of potentized dispersions of each of the identified allergens and the method of preparation for the same. The present disclosure further relates to a kit consisting of at least one allergen, at least one vehicle and accessories which include bottles and mixing sticks.

### FIELD OF DISCLOSURE

The present disclosure relates to a medicinal formulation.

Particularly, the present disclosure relates to an anti-allergic formulation. More particularly, the present disclosure relates to the anti-allergic formulation for treating, controlling, reducing or preventing allergy.

### BACKGROUND

Allergy is an over-reaction of the body's immune system to foreign substances (known as allergens). The inflammatory responses mediated by the immune system are typically classified into four categories: I, II, II, and IV. Allergic responses belong to reactions of Type I immediate hypersensitivity in which a person's body is hyper-sensitized and develops IgE-type antibodies to typical allergens. Mast cells are a key component in the cascade of allergic responses. Mast cells are resident cells of connective tissues and contain many different allergenic compounds in their cytoplasmic granules, the best known of which is histamine.

Immediate hypersensitivity results from the following sequence of events: production of IgE by B cells in response to an allergen, binding of the IgE molecules to Fc epsilon RI (FcεRI) receptors on the surface of mast cells, interaction of a later-introduced allergen of the same type with the bound IgE and activation of the mast cells and release of mediators of allergy (a process known as degranulation), including histamine. Histamine dilates blood vessels, renders them leaky and activates the endothelium. This leads to local edema, warmth, redness and the attraction and accumulation of other inflammatory cells to the site of the release of histamine Histamine also irritates nerve endings (leading to itching or pain).

Other inflammatory mediators released by activated mast cells include prostaglandins (such as prostaglandin D₂ (“PGDi”), prostaglandin E₂ (“PGE₂”), and prostaglandin F₂ (“PGF₂”), leukotrienes (such as leukotriene C₄ (“LTC4”), leukotriene D₄ (“LTD₄”), leukotriene E₄ (“LTE₄”)), chemoattractants (such as platelet activating factors (“PAFs”), glycoprotein (Cs₃), and cytokines (such as BL-I, IL-4, IL-6, and TNF-α). PAFs are potent chemoattractants and stimuli of lysosomal enzyme release and reactive oxygen product formation by neutrophils, eosinophils, and macrophages. In addition, PAFs increase the stickiness of endothelial cells for leukocytes, promoting their accumulation at the site of inflammation. Cs₃ is a powerful chemoattractant for eosinophils. TNF-α is a major proinflammatory cytokine, the activities of which include chemotaxis for eosinophils. The recruited eosinophils in turn secrete many cytokines such as IL-3, GM-CSF (granulocyte-macrophage colony-stimulating factor), TNF-α, and IL-I when activated. Any of these cytokines serves to enhance and sustain the allergic inflammatory process. For example, EL-3 secreted by eosinophils can serve as a growth factor for mast cells, and thus enhance a release of proinflammatory compounds from mast cells. Therefore, an uncontrolled amplification of the allergic inflammatory process quickly can become damaging to the host tissue surrounding the site of inflammation.

Allergy is a complex disease. Many factors contribute to the sensitization event. Among these is the susceptibility of the individual defined by an as yet insufficiently understood interplay between several genes. Another important factor is allergen exposure above certain thresholds. Several environmental factors may be important in the sensitization process including pollution, childhood infections, parasite infections, intestinal microorganisms, etc. Once an individual is sensitized and the allergic immune response established, the presence of only minute amounts of allergen is efficiently translated into symptoms. The natural course of allergic disease is usually accompanied by aggravation at two levels. Firstly, a progression of symptoms and disease severity, as well as disease progression, for example from hay fever to asthma. Secondly, dissemination in offending allergens most often occurs resulting in allergic multi-reactivity. Chronic inflammation leads to a general weakening of the mucosal defense mechanisms resulting in unspecific irritation and eventually destruction of the mucosal tissue. Infants may become sensitized primarily to foods, i.e. milk, resulting in eczema or gastrointestinal disorders; however, most often they outgrow these symptoms spontaneously. These infants are at risk of developing inhalation allergy later in their lives.

The most important allergen sources are found among the most prevalent particles of a certain size in the air which we breathe. These sources are remarkably universal and include grass pollen and house dust mite faecal particles, which together are responsible for approximately 50% of all allergies. It also includes animal dander, i.e. cat and dog dander, other pollen, such as mugwort pollen, and micro-fungi, such as Alternaria are important.

The allergic reaction is prompt and peaks within 20 minutes upon contact with the offending allergen. Furthermore, the allergic reaction is specific in the sense that a particular individual is sensitized to particular allergen(s), whereas the individual does not necessarily show an allergic reaction to other substances known to cause allergic disease. The allergic phenotype is characterized by a pronounced inflammation of the mucosa of the target organ and by the presence of allergen specific antibody of the IgE class in the circulation and on the surfaced of mast-cells and basophils.

Allergy disease management comprises diagnosis and treatment including prophylactic treatments. Diagnosis of allergy is concerned with the demonstration of allergen specific IgE and identification of the allergen source. In many cases a careful anamnesis may be sufficient for the diagnosis of allergy and for the identification of the offending allergen source material. Most often, however, the diagnosis is supported by objective measures, such as skin prick test, blood test or provocation test.

The therapeutic options fall in three major categories. The first opportunity is allergen avoidance or reduction of the exposure. Whereas allergen avoidance is obvious e.g. in the case of food allergens, it may be difficult or expensive, as for house dust mite allergens, or it may be impossible, as for pollen allergens. The second and most widely used therapeutic option is the prescription of classical symptomatic drugs like anti-histamines, mast cell stabilizers, non-steroidal anti-inflammatory drugs (“NSAIDs”) for moderate cases and glucocorticoids for more severe cases.

Glucocorticoids represent one of the most effective clinical treatments for a range of inflammatory conditions, including acute inflammation. However, steroidal drugs can have side effects that threaten the overall health of the patient. Chronic administration of glucocorticoids can lead to drug-induced osteoporosis by suppressing intestinal calcium absorption and inhibiting bone formation. Other adverse side effects of chronic administration of glucocorticoids include hypertension, hyperglycemia, hyperlipidemia and hypercholesterolemia because of the effects of these drugs on the body metabolic processes.

These pharmacological treatments are merely symptomatic and have no long lasting beneficial effect on the course of the allergy itself. Further, symptomatic drugs are not safe & efficient and they do not alter the natural cause of the disease, neither do they control the disease dissemination. They provide only temporary and symptomatic relief at the site of application.

Furthermore, with conventional subcutaneous desensitization, the duration of treatment is around 3-5 years and usually comprises around 30-100 allergen injections. As high allergen doses have to be injected, allergic side effects may occur and patients must stay under medical supervision for at least 1 hour.

Therefore, there is a continued need to provide formulations and methods for treating, controlling, reducing, ameliorating, or preventing allergy with lower allergen doses. In addition, it is also desirable to provide such formulations and methods that have no or very low levels of side effects. Still further there is a need for a formulation that can induce protective immunity against allergy or allergic infections, in a re-producible manner and which has better safety profile than vaccines.

### Objects of the Present Disclosure

One of the objects of the present disclosure is to provide formulations for treating, controlling, reducing, ameliorating or preventing allergy.

Yet another object of the present disclosure is to provide formulations which have curative effects.

A further objective of the present disclosure is to provide formulations which have improved therapeutic efficacy.

Still further object of the present disclosure is to provide formulations which are administered in low dose.

Another object of the present disclosure is to provide formulations without side effects.

Yet another object of the present disclosure is to provide formulations which are easy to manufacture.

Still another object of the present disclosure is to provide a method for preparation of novel formulations, potentized by means of a mathematico-mechanical process for maximal therapeutic benefits.

Still another object of the present disclosure is to provide a method for treating, controlling, reducing or preventing allergy.

### SUMMARY

In accordance with one aspect of the present disclosure there is provided a method for the treatment of allergic conditions; said method comprising the following steps:
a. obtaining the allergy profile of an individual, identifying allergens to which the individual is susceptible and the level of susceptibility;
b. arranging the identified allergen according to the sensitivity;
c. diluting 1 μg to 1 mg mass of each of the identified allergen individually with at least one vehicle to obtain a primary dilution of 1c potency;
d. serially diluting with potentization, each of the primary dilutions with the vehicle in a ratio of 1:9 to 50:50 to obtain potentized dispersions for each of the identified allergens; characterized in that the potencies of said potentized dispersions are in the range of 2c to 100000c; preferably in the range of 30c to 500c;
e. mixing the potentized dispersions of each of the identified allergens to obtain a formulation; characterized in that the potency of each of said dispersions is directly proportional to the sensitivity of the individual to the identified allergen;
f. administering a predetermined amount of the formulation having a predetermined potency to the individual for a predetermined period;
g. retesting the allergy profile of the individual after a predetermined period; and
h. repeating at least once the method steps of (a) to (f) after readjusting the potencies of the individually identified allergens depending upon the retested allergy profile, until a significant reduction in said allergic conditions is observed.

Typically, the allergen is at least one selected from the group consisting of pollen, animal dander, fungal spores, dust, venoms, food, biochemical allergens and chemical allergens.

Typically, the vehicle is at least one selected from the group consisting of normal saline, distilled water and ethyl alcohol.

Typically, the formulation is in a wet dosage form.

Typically, the amount of the formulation administered per day is in the range of 0.01 ml to 1 ml.

In accordance with another aspect of the present disclosure there is provided a formulation comprising potentized dispersions of each of the identified allergens to which an individual is susceptible; characterized in that the potency of each of said potentized dispersions is directly proportional to the sensitivity of the individual to the identified allergen.

Typically, the allergen is at least one selected from the group consisting of pollen, animal dander, fungal spores, dust, venoms, food, biochemical allergens and chemical allergens.

Typically, the potency of the potentized dispersion is in the range of 2c to 100000c; preferably, in the range of 30c to 500c.

Typically, the potentized dispersion comprises a vehicle and the allergens to which an individual is susceptible; wherein the vehicle is at least one selected from the group consisting of normal saline, distilled water and ethyl alcohol.

Typically, the formulation is in a wet dosage form.

In accordance with yet another aspect of the present disclosure there is provided a process for preparing a formulation; said process comprising:
a. obtaining allergens to which an individual is susceptible;
b. diluting 1 μg to 1 mg mass of each of the identified allergen individually with at least one vehicle to obtain a primary dilution of 1c potency;
c. serially diluting with potentization, each of the primary dilutions with the vehicle in a ratio of 1:9 to 50:50 to obtain potentized dispersions for each of the identified allergens; characterized in that the potencies of said potentized dispersions are in the range of 2c to 100000c; preferably, in the range of 30c to 500c; and
d. mixing the potentized dispersions to obtain a formulation; characterized in that the potency of each of said dispersions is directly proportional to the sensitivity of the individual to the identified allergens.

Typically, the allergen is at least one selected from the group consisting of pollen, animal dander, fungal spores, dust, venoms, food, biochemical allergens and chemical allergens.

Typically, the vehicle is at least one selected from the group consisting of normal saline, distilled water and ethyl alcohol.

Typically, the formulation is in a wet dosage form.

Typically, the method step of potentization is carried out by exerting a force of at least 6 dynes rhythmically at a frequency of 10 strokes in two minutes.

In accordance with still another aspect of the present disclosure there is provided a kit comprising (a) at least one allergen, (b) at least one vehicle and (c) accessories which include bottles and mixing sticks.

Typically, the allergen is at least one selected from the group consisting of pollen, animal dander, fungal spores, dust, venoms, food, biochemical allergens and chemical allergens.

Typically, the vehicle is at least one selected from the group consisting of normal saline, distilled water and ethyl alcohol.

### DETAILED DESCRIPTION

Conventional methods for the management or the treatment of an allergy are symptomatic, which mainly includes use of anti-histamines, mast cell stabilizers, non-steroidal anti-inflammatory drugs (“NSAIDs”) and glucocorticoids. These methods are merely symptomatic and have no long lasting beneficial effect on the course of the allergy itself. Further, these are not safe & efficient and they do not alter the natural cause of the disease, neither do they control the disease dissemination. They provide only temporary and symptomatic relief at the site of application. Therefore, the inventor of the present disclosure developed a method for the treatment of the allergic conditions.

In accordance with one aspect of the present disclosure there is provided a method for the treatment of allergic conditions; said method comprising the following steps:

In the first step, the allergy profile of an individual is obtained in order to identify allergens to which the individual is susceptible and level of susceptibility. The identified allergens are then arranged in a decreasing order of their sensitivity.

Typically, the allergen is at least one selected from the group consisting of pollen, animal dander, fungal spores, dust, venoms, food, biochemical allergens and chemical allergens.

In the second step, each of the identified allergen is diluted individually with at least one vehicle to obtain primary dilutions of 1c potency. Typically mass of the identified allergen is in the range of 1 μg to 1 mg. The primary dilutions so obtained are then serially diluted individually with potentization, by using the vehicle in a ratio of 1:9 to 50:50 to obtain potentized dispersions for each of the identified allergens having potency in the range of 2c to 100000c.

Typically, the vehicle is at least one selected from the group consisting of normal saline, distilled water and ethyl alcohol.

In accordance with one of the exemplary embodiment of the present disclosure the potency of the potentized dispersions is maintained in the range of 30c to 500c.

In the third step, the potentized dispersions of each of the identified allergens are mixed to obtain a formulation. The potency of the potentized dispersion is directly proportional to the sensitivity of the individual to the identified allergens.

In the fourth step, the formulation is then administered at a predetermined amount and at predetermined potencies to the individual for the predetermined period. The individual is then retested after the predetermined period in order to obtain a next allergy profile.

Typically, the amount of the formulation administered per day is in the range of 0.01 ml to 1 ml.

As per the next allergy profile and the altered susceptibility of the individual to the allergens, aforementioned steps are repeated after readjusting the potencies of the individually identified allergens.

Typically, the aforementioned steps are repeated until the significant reduction in the allergic conditions of the individual.

Typically, the formulation is in a wet dosage form.

In accordance with another aspect of the present disclosure there is provided a formulations which interferes with basic immunological mechanisms and result in persistent reduction of the individual's allergic conditions.

The formulation the present disclosure is typically a wet dosage form and it comprises potentized dispersions of allergens to which an individual is susceptible.

An allergen is any compound, substance, or material which is capable of evoking an allergic reaction. For carrying out the present disclosure, the allergen may be selected, inter alia, from natural or native allergens, modified natural allergens, synthetic allergens, allergen extracts, purified fractions of an allergen extract, modified allergens, recombinant allergens, mutants of a recombinant allergen, allergoids, and mixtures or combinations thereof.

Examples of allergens include compounds or compound mixtures representing or being obtained from pollen, animal dander, fungal spores, dust, venoms, food, biochemical allergen and chemical allergen. The allergens or allergen mixtures may be used in their native form, or after physical or chemical modification or derivatisation.

The biochemical allergen and chemical allergen includes native or recombinant proteins or peptides, fragments or truncated versions of native or recombinant proteins or peptides, fusion proteins, synthetic compounds (chemical allergens), synthetic compounds which mimic an allergen, or chemically or physically altered allergens, such as allergens modified by heat denaturation.

Examples of grass pollen allergens include but are not limited to pollen allergens from maize, Timothy grass, meadow grass, Bermuda grass, bluegrass, brome, paspalum, orchard grass, perennial rye, sweet vernal, meadow fescue, velvet, wild oat, perennial rye, common reed, June (Kentucky blue), red top, Johnson, cultivated rye, cultivated oat, cultivated wheat, meadow foxtail, Bahia, wild rye, Canary grass, couch, Sudan grass, salt grass and any mixture thereof.

Examples of weed pollen allergens include but are not limited to allergens from common ragweed, Western ragweed, giant ragweed, false ragweed, wormwood, ox-eye daisy, Russian thistle, golden rod, mugwort, pellitory, nettles, plantain, duck weed, fat hen, sorrel, pigweed, goosefoot, dandelion, goldenrod, helianthus, sage, cocklebur, clover, alfalfa, rabbitbush, careless weed, saltbush, poverty weed, rough pigweed, yellow dock, dog fennel and any mixtures of these.

Trees from which pollen allergens may be obtained to carry out the disclosure include but are not limited to alder, elm, olive, ash, hazel, pine, beech, heath, plane, birch, hickory, poplar, chestnut, hornbeam, lime, linden, maple, ti, cypress, myrtle, wattle, Japanese cedar, mulberry, walnut, Western red cedar, oak, willow and any mixtures of these. Optionally, the allergens may represent mixtures of grass-, weed-, and/or tree pollen allergens.

In another embodiments the allergens fungi spores, such as mold spores, such as from Penicillium n., Cladosporium h., Aspergillus f., Mucor r., Candida a., Altemaria a., Botrytis c, Helminthosporium h., Fusarium m., Fusarium s., Stemphylium b., Rhizopus n., Aureobasidium p. (Pullularia), Phoma b., Epicoccum p., Trichoderma v., Curvularia s., Trichophyton m., Grass Smut, Malassezia pachydermatis, Cephalothecium, Hormodendrum, Mucor, Rhisopus, or any mixtures thereof.

In accordance with still another embodiment the allergens are based on animal venoms, such as venoms from the honeybee, yellow jacket, wasp, paper wasp, yellow hornet, cockroach, flea, deer fly, black ant, housefly, red ant, mosquito, fire ant, moth, horse fly or any mixtures thereof.

A further group of allergens are based on other animal products which are not venoms, in particular animal hair, animal dander, excretions of (house) dust mites, calyx components of cockroaches and the like.

In accordance with yet another embodiment the allergens are natural allergens which are not airborne, but typically encountered in food and which include but are not limited to allergens from peanuts, nuts, sesame, seafood, milk, egg, peas, beans, soy beans, other legumes, wheat, maize or any mixtures thereof.

The potency of the potentized dispersions in the formulation of the present disclosure is directly proportional to the sensitivity or susceptibility of the individual to the identified allergen. Typically the potency of the potentized dispersions is maintained in the range of 2c to 100000c.

In accordance with one of the exemplary embodiment of the present disclosure the potency of the potentized dispersions is maintained in the range of 30c to 500c.

The vehicle present in the potentized dispersion includes normal saline, distilled water and ethyl alcohol.

In accordance with yet another aspect of the present disclosure there is provided a process for preparing a formulation; said process comprising the following steps:

In the first step, the allergens to which an individual is susceptible are obtained and then each of the identified allergen is diluted individually with at least one vehicle to obtain primary dilutions of 1c potency. Typically, the mass of the identified allergen is in the range of 1 μg to 1 mg. The primary dilutions so obtained are then serially diluted with potentization, by using the vehicle in a ratio of 1:9 to 50:50 to obtain potentized dispersions for each of the identified allergens having potency in the range of 2c to 100000c.

In accordance with one of the exemplary embodiment of the present disclosure the potency of the potentized dispersions is maintained in the range of 30c to 500c.

In the second step, the potentized dispersions are mixed to obtain a formulation. The potency of the potentized dispersion is directly proportional to the sensitivity of the individual to the identified allergens.

Typically, the potentization is effected by stroking by holding a bottle containing the dispersion in a closed first and striking the first on a hard surface repeatedly at a regular frequency or by exercising similar powerful stroke using a mechanical device which can strike a bottle on a hard surface.

Typically, strokes are given about 10 times.

Typically, the bottle containing the preparation for stroking for potentization is a securely stoppered glass bottle.

Typically, a mechanical device is used to exert a force of at least 6 dynes rhythmically at a frequency of 10 strokes in two minutes.

In accordance with still another aspect of the present disclosure there is provided a kit for preparing a formulation of the present disclosure. The kit comprises (a) at least one allergen, (b) at least one vehicle and (c) accessories which include bottles and mixing sticks.

Typically, the allergen includes pollen, animal dander, fungal spores, dust, venoms, food, biochemical allergens and chemical allergens.

Typically, the vehicle includes normal saline, distilled water and ethyl alcohol.

Hereinafter, the present disclosure will be described in more detail with reference to the following examples, but the scope of the present disclosure is not limited thereto.

### Example 1

2 μg of dust allergens were mixed with 90 ml of ethyl alcohol (90 to 100%) to obtain a primary dilution of 1c potency. The primary dilution so obtained was then serially diluted with potentization, by using the vehicle in a ratio of 1:9 to 50:50 to obtain a potentized dispersion having potency in the range of 2c to 100000c. The potentization was effected by holding and subsequently stroking a bottle containing the dispersion in a closed first and striking the first on a hard surface repeatedly at a regular frequency or by exercising similar powerful stroke using a mechanical device which can strike a bottle on a hard surface. The mechanical device was adopted to exert a force of at least 6 dynes rhythmically at a frequency of 10 strokes in two minutes.

### Example 2

1 μg of pollen allergens were mixed with 90 ml of ethyl alcohol (90 to 100%) to obtain a primary dilution of 1c potency. The primary dilution so obtained was then serially diluted with potentization, by using the vehicle in a ratio of 1:9 to 50:50 to obtain a potentized dispersion having potency in the range of 2c to 100000c. The potentization was effected by holding and subsequently stroking a bottle containing the dispersion in a closed first and striking the first on a hard surface repeatedly at a regular frequency or by exercising similar powerful stroke using a mechanical device which can strike a bottle on a hard surface. The mechanical device was adopted to exert a force of at least 6 dynes rhythmically at a frequency of 10 strokes in two minutes.

### Example 3

1.5 μg of food allergens were mixed with 90 ml of ethyl alcohol (90 to 100%) to obtain a primary dilution of 1c potency. The primary dilution so obtained was then serially diluted with potentization, by using the vehicle in a ratio of 1:9 to 50:50 to obtain a potentized dispersion having potency in the range of 2c to 100000c. The potentization was effected by holding and subsequently stroking a bottle containing the dispersion in a closed first and striking the first on a hard surface repeatedly at a regular frequency or by exercising similar powerful stroke using a mechanical device which can strike a bottle on a hard surface. The mechanical device was adopted to exert a force of at least 6 dynes rhythmically at a frequency of 10 strokes in two minutes.

### Anecdotal Studies:

1) A 35 years old female, presented with the suite of symptoms. Her condition was directed as allergic inflammatory reactions. Therefore, in accordance with this disclosure her allergic profile was identified. Her allergy profile was as follows:

| Sr. | | IgE profile | Reference Range |
|---|---|---|---|
| No. | Allergen | (kUA/L) | (kUA/L) |
| 1 | MILK | 0.07 | <0.1 Negative |
| 2 | RICE | 0.10 | >0.1 Positive |
| 3 | WHEAT | 0.09 | <0.1 Negative |
| 4 | DOG DANDER | 0.07 | >0.1 Positive |
| 5 | EGG WHITE | 0.08 | <0.1 Negative |
| 6 | CHEESE | 0.02 | >0.1 Positive |
| | CHEDDAR | | |
| 7 | GREEN PEA | 0.03 | <0.1 Negative |
| 8 | COCONUT | 0.08 | >0.1 Positive |
| 9 | GARLIC | H 0.23 | <0.1 Negative |
| 10 | GLUTEN | 0.03 | >0.1 Positive |
| 11 | EGG YOLK | 0.04 | <0.1 Negative |
| 12 | PEANUT | H 0.13 | >0.1 Positive |
| 13 | CABBAGE | 0.02 | <0.1 Negative |
| 14 | CASHEW | 0.02 | >0.1 Positive |
| 15 | BANANA | H 0.19 | <0.1 Negative |
| 16 | CORN | 0.09 | >0.1 Positive |
| 17 | CAULIFLOWER | 0.04 | <0.1 Negative |
| 18 | POTATO | 0.03 | >0.1 Positive |
| 19 | SILK | 0.04 | <0.1 Negative |
| 20 | COFFEE | 0.02 | >0.1 Positive |
| 21 | LEMON | 0.05 | <0.1 Negative |
| 22 | MUSTARD | 0.03 | >0.1 Positive |
| 23 | APPLE | 0.03 | <0.1 Negative |
| 24 | ONION | 0.10 | >0.1 Positive |
| 25 | TOMATO | H 0.30 | <0.1 Negative |
| 26 | CARROT | 0.06 | >0.1 Positive |
| 27 | PLUM | 0.03 | <0.1 Negative |
| 28 | VELVET | 0.05 | >0.1 Positive |
| 29 | LENTIL | 0.05 | <0.1 Negative |
| | Total IgE | 215.9 | <1.5 −378 IU/ml |

The aforementioned allergy profile indicated that she was allergic to tomato, garlic, peanut and banana. It was also observed that she was more allergic to tomato as compared to garlic, peanut and banana.

As per her allergy profile a suitable formulation was designed. The formulation contained a mixture of potentized dispersions of tomato, garlic, peanut and banana allergens having potency of 400c, 200c, 150c and 100c respectively. The dose of the formulation was 0.2 ml, 3 times a day for 2 weeks.

After 15 days her allergy profile was again identified.

| Sr. | | IgE profile | Reference Range |
|---|---|---|---|
| No. | Allergen | (kUA/L) | (kUA/L) |
| 1 | MILK | 0.06 | <0.1 Negative |
| 2 | RICE | 0.11 | >0.1 Positive |
| 3 | WHEAT | 0.09 | <0.1 Negative |
| 4 | DOG DANDER | 0.05 | >0.1 Positive |
| 5 | EGG WHITE | 0.09 | <0.1 Negative |
| 6 | CHEESE | 0.02 | >0.1 Positive |
| | CHEDDAR | | |
| 7 | GREEN PEA | 0.04 | <0.1 Negative |
| 8 | COCONUT | 0.07 | >0.1 Positive |
| 9 | GARLIC | H 0.12 | <0.1 Negative |
| 10 | GLUTEN | 0.03 | >0.1 Positive |
| 11 | EGG YOLK | 0.03 | <0.1 Negative |
| 12 | PEANUT | 0.01 | >0.1 Positive |
| 13 | CABBAGE | 0.03 | <0.1 Negative |
| 14 | CASHEW | 0.02 | >0.1 Positive |
| 15 | BANANA | 0.05 | <0.1 Negative |
| 16 | CORN | 0.09 | >0.1 Positive |
| 17 | CAULIFLOWER | 0.04 | <0.1 Negative |
| 18 | POTATO | 0.02 | >0.1 Positive |
| 19 | SILK | 0.05 | <0.1 Negative |
| 20 | COFFEE | 0.02 | >0.1 Positive |
| 21 | LEMON | 0.05 | <0.1 Negative |
| 22 | MUSTARD | 0.03 | >0.1 Positive |
| 23 | APPLE | 0.03 | <0.1 Negative |
| 24 | ONION | 0.10 | >0.1 Positive |
| 25 | TOMATO | H 0.15 | <0.1 Negative |
| 26 | CARROT | 0.06 | >0.1 Positive |
| 27 | PLUM | 0.02 | <0.1 Negative |
| 28 | VELVET | 0.02 | >0.1 Positive |
| 29 | LENTIL | 0.02 | <0.1 Negative |
| | Total IgE | 152.2 | <1.5 −378 IU/ml |

The aforementioned allergy profile indicated that, IgE level for allergens such as tomato, garlic, peanut and banana has been reduced indicating effectiveness of the present formulation. It was also observed that IgE level for allergens such as tomato and garlic still need to be reduced.

Accordingly, a suitable formulation was designed. The formulation contained a mixture of potentized dispersions of tomato and garlic allergens having potency of 200c and 100c respectively. The dose of the formulation was 0.1 ml 3 times a day for 2 weeks. It was found that there is significant reduction in IgE profile after the treatment.

After continuing the same therapy for next 10 weeks, there was 90% reduction in inflammatory reactions.

2) Female 41 years old, presented with the complaints of allergic rhinitis. Her symptoms were severe sneezes followed by mucous discharge from nose. The episode would occur once a week. There would be itching all over the face and nose. She was sensitive to odors and there was burning in eyes. She would take antihistaminic regularly.

Therefore, in accordance with this disclosure her allergic profile was identified. Her allergy profile was as follows:

| Sr. | | IgE profile | Reference Range |
|---|---|---|---|
| No. | Allergen | (kUA/L) | (kUA/L) |
| 1 | MILK | 0.1 | <0.1 Negative |
| 2 | RICE | 0.1 | >0.1 Positive |
| 3 | WHEAT | H 0.25 | <0.1 Negative |
| 4 | ANIMAL | H 0.26 | >0.1 Positive |
| | DANDER | | |
| 5 | EGG WHITE | 0.09 | <0.1 Negative |
| 6 | CHEESE | 0.09 | >0.1 Positive |
| | CHEDDAR | | |
| 7 | GREEN PEA | 0.1 | <0.1 Negative |
| 8 | COCONUT | 0.1 | >0.1 Positive |
| 9 | GARLIC | 0.08 | <0.1 Negative |
| 10 | GLUTEN | 0.06 | >0.1 Positive |
| 11 | EGG YOLK | 0.09 | <0.1 Negative |
| 12 | PEANUT | H 0.32 | >0.1 Positive |
| 13 | CABBAGE | H 0.23 | <0.1 Negative |
| 14 | CASHEW | 0.09 | >0.1 Positive |
| 15 | BANANA | 0.08 | <0.1 Negative |
| 16 | CORN | 0.07 | >0.1 Positive |
| 17 | CAULIFLOWER | 0.09 | <0.1 Negative |
| 18 | POTATO | 0.08 | >0.1 Positive |
| 19 | SILK | 0.1 | <0.1 Negative |
| 20 | COFFEE | 0.1 | >0.1 Positive |
| 21 | LEMON | 0.09 | <0.1 Negative |
| 22 | MUSTARD | 0.09 | >0.1 Positive |
| 23 | APPLE | 0.08 | <0.1 Negative |
| 24 | ONION | 0.1 | >0.1 Positive |
| 25 | TOMATO | 0.09 | <0.1 Negative |
| 26 | CARROT | 0.1 | >0.1 Positive |
| 27 | PLUM | 0.08 | <0.1 Negative |
| 28 | VELVET | 0.1 | >0.1 Positive |
| 29 | LENTIL | 0.09 | <0.1 Negative |
| 30 | DUST | H 0.35 | >0.1 Positive |
| 31 | POLLEN | H 0.27 | >0.1 Positive |
| | Total IgE | 392 | <1.5 −378 IU/ml |

Her allergy profile indicated that she was allergic to wheat, animal dander, peanut, cabbage, pollen and dust.

As per her allergy profile a suitable formulation was designed. The formulation contained a mixture of potentized dispersions of pollen, peanut, dust, animal dander, wheat and cabbage allergens having potency of 600c, 550c, 300c, 350c, 150c and 100c respectively. The dose of the formulation was 0.2 ml, 3 times a day for 2 weeks.

After 15 days her allergy profile was again identified.

Her allergy profile was as follows:

| Sr. | | IgE profile | Reference Range |
|---|---|---|---|
| No. | Allergen | (kUA/L) | (kUA/L) |
| 1 | MILK | 0.09 | <0.1 Negative |
| 2 | RICE | 0.05 | >0.1 Positive |
| 3 | WHEAT | H 0.15 | <0.1 Negative |
| 4 | ANIMAL | 0.1 | >0.1 Positive |
| | DANDER | | |
| 5 | EGG WHITE | 0.09 | <0.1 Negative |
| 6 | CHEESE | 0.09 | >0.1 Positive |
| | CHEDDAR | | |
| 7 | GREEN PEA | 0.05 | <0.1 Negative |
| 8 | COCONUT | 0.1 | >0.1 Positive |
| 9 | GARLIC | 0.09 | <0.1 Negative |
| 10 | GLUTEN | 0.06 | >0.1 Positive |
| 11 | EGG YOLK | 0.09 | <0.1 Negative |
| 12 | PEANUT | H 0.25 | >0.1 Positive |
| 13 | CABBAGE | H 0.15 | <0.1 Negative |
| 14 | CASHEW | 0.09 | >0.1 Positive |
| 15 | BANANA | 0.08 | <0.1 Negative |
| 16 | CORN | 0.08 | >0.1 Positive |
| 17 | CAULIFLOWER | 0.09 | <0.1 Negative |
| 18 | POTATO | 0.08 | >0.1 Positive |
| 19 | SILK | 0.09 | <0.1 Negative |
| 20 | COFFEE | 0.1 | >0.1 Positive |
| 21 | LEMON | 0.04 | <0.1 Negative |
| 22 | MUSTARD | 0.09 | >0.1 Positive |
| 23 | APPLE | 0.08 | <0.1 Negative |
| 24 | ONION | 0.09 | >0.1 Positive |
| 25 | TOMATO | 0.08 | <0.1 Negative |
| 26 | CARROT | 0.1 | >0.1 Positive |
| 27 | PLUM | 0.08 | <0.1 Negative |
| 28 | VELVET | 0.1 | >0.1 Positive |
| 29 | LENTIL | 0.04 | <0.1 Negative |
| 30 | DUST | H 0.23 | >0.1 Positive |
| 31 | POLLEN | H 0.11 | >0.1 Positive |
| | Total IgE | 301 | <1.5 −378 IU/ml |

The aforementioned allergy profile indicated that, IgE level for the allergens such as pollen, peanut, dust, animal dander, wheat and cabbage has been reduced indicating effectiveness of the present formulation. It was also observed that IgE level for allergens such as pollen, peanut, dust, wheat and cabbage still need to be reduced.

Accordingly, a suitable formulation was designed. The formulation contained a mixture of potentized dispersions of pollen, peanut, dust, wheat and cabbage allergens having potency of 450c, 350c, 150c, 100c and 50c respectively. The dose of the formulation was 0.1 ml 3 times a day for 2 weeks.

After continuing the same therapy for next 12 weeks, there was 80% reduction in her symptoms of severe sneezes followed by mucous discharges from nose.

3) Male 20 years old, presented with complaints of frequent colds. He could get nose block and thick discharge from nose. He was getting cold once in a month, lasting for one week.

Therefore, in accordance with this disclosure his allergy profile was identified. His allergy profile was as follows:

| Sr. | | IgE profile | Reference Range |
|---|---|---|---|
| No. | Allergen | (kUA/L) | (kUA/L) |
| 1 | MILK | 0.10 | <0.1 Negative |
| 2 | RICE | 0.11 | >0.1 Positive |
| 3 | WHEAT | 0.10 | <0.1 Negative |
| 4 | ANIMAL | 0.10 | >0.1 Positive |
| | DANDER | | |
| 5 | EGG WHITE | 0.06 | <0.1 Negative |
| 6 | CHEESE | 0.03 | >0.1 Positive |
| | CHEDDAR | | |
| 7 | GREEN PEA | 0.09 | <0.1 Negative |
| 8 | COCONUT | 0.05 | >0.1 Positive |
| 9 | GARLIC | 0.02 | <0.1 Negative |
| 10 | GLUTEN | 0.06 | >0.1 Positive |
| 11 | EGG YOLK | 0.02 | <0.1 Negative |
| 12 | PEANUT | H 0.14 | >0.1 Positive |
| 13 | CABBAGE | 0.03 | <0.1 Negative |
| 14 | CASHEW | 0.09 | >0.1 Positive |
| 15 | BANANA | 0.05 | <0.1 Negative |
| 16 | CORN | 0.08 | >0.1 Positive |
| 17 | CAULIFLOWER | 0.03 | <0.1 Negative |
| 18 | POTATO | 0.07 | >0.1 Positive |
| 19 | SILK | 0.05 | <0.1 Negative |
| 20 | COFFEE | 0.02 | >0.1 Positive |
| 21 | LEMON | 0.06 | <0.1 Negative |
| 22 | MUSTARD | 0.09 | >0.1 Positive |
| 23 | APPLE | 0.07 | <0.1 Negative |
| 24 | ONION | 0.01 | >0.1 Positive |
| 25 | TOMATO | 0.04 | <0.1 Negative |
| 26 | CARROT | 0.06 | >0.1 Positive |
| 27 | PLUM | 0.01 | <0.1 Negative |
| 28 | VELVET | 0.02 | >0.1 Positive |
| 29 | LENTIL | 0.04 | <0.1 Negative |
| 30 | DUST | 0.10 | >0.1 Positive |
| 31 | POLLEN | H 0.21 | >0.1 Positive |
| | Total IgE | 201 | <1.5 −378 IU/ml |

His allergy profile indicated that he was more allergic to pollen as compared to peanuts.

As per the aforementioned allergy profile a suitable formulation was designed. The formulation contained a mixture of potentized dispersions of pollen and peanut allergens having potency of 250c and 150c respectively. The dose of the formulation was 0.2 ml 3 times a day for 2 weeks.

After 15 days his allergy profile was again identified.

| Sr. | | IgE profile | Reference Range |
|---|---|---|---|
| No. | Allergen | (kUA/L) | (kUA/L) |
| 1 | MILK | 0.07 | <0.1 Negative |
| 2 | RICE | 0.10 | >0.1 Positive |
| 3 | WHEAT | 0.10 | <0.1 Negative |
| 4 | ANIMAL | 0.5 | >0.1 Positive |
| | DANDER | | |
| 5 | EGG WHITE | 0.01 | <0.1 Negative |
| 6 | CHEESE | 0.04 | >0.1 Positive |
| | CHEDDAR | | |
| 7 | GREEN PEA | 0.04 | <0.1 Negative |
| 8 | COCONUT | 0.07 | >0.1 Positive |
| 9 | GARLIC | 0.02 | <0.1 Negative |
| 10 | GLUTEN | 0.05 | >0.1 Positive |
| 11 | EGG YOLK | 0.02 | <0.1 Negative |
| 12 | PEANUT | 0.08 | >0.1 Positive |
| 13 | CABBAGE | 0.03 | <0.1 Negative |
| 14 | CASHEW | 0.02 | >0.1 Positive |
| 15 | BANANA | 0.02 | <0.1 Negative |
| 16 | CORN | 0.06 | >0.1 Positive |
| 17 | CAULIFLOWER | 0.03 | <0.1 Negative |
| 18 | POTATO | 0.02 | >0.1 Positive |
| 19 | SILK | 0.05 | <0.1 Negative |
| 20 | COFFEE | 0.02 | >0.1 Positive |
| 21 | LEMON | 0.01 | <0.1 Negative |
| 22 | MUSTARD | 0.03 | >0.1 Positive |
| 23 | APPLE | 0.05 | <0.1 Negative |
| 24 | ONION | 0.01 | >0.1 Positive |
| 25 | TOMATO | 0.03 | <0.1 Negative |
| 26 | CARROT | 0.03 | >0.1 Positive |
| 27 | PLUM | 0.02 | <0.1 Negative |
| 28 | VELVET | 0.02 | >0.1 Positive |
| 29 | LENTIL | 0.02 | <0.1 Negative |
| 30 | DUST | 0.10 | >0.1 Positive |
| 31 | POLLEN | H 0.15 | >0.1 Positive |
| | Total IgE | 182 | <1.5 −378 IU/ml |

The aforementioned allergy profile indicated that, IgE level for the allergens such as pollen and peanut has been reduced indicating effectiveness of the present formulation. It was also observed that IgE level for pollen allergens still need to be reduced.

Accordingly, a suitable formulation was designed. The formulation contained a mixture of potentized dispersions of pollen allergens having potency of 50c. The dose of the formulation was 0.1 ml 3 times a day for 2 weeks.

4) Male 10 years old, presented with the complaints of frequent colds. He could get nose block and thick discharge from nose. He was getting running nose and sneezing once in 15 days, each episode lasting for 3-2 days. Nasal discharge was watery along with irritation in nose.

Therefore, in accordance with this disclosure his allergy profile was identified. His allergy profile was as follows:

| Sr. | | IgE profile | Reference Range |
|---|---|---|---|
| No. | Allergen | (kUA/L) | (kUA/L) |
| 1 | MILK | 0.05 | <0.1 Negative |
| 2 | RICE | 0.10 | >0.1 Positive |
| 3 | WHEAT | 0.11 | <0.1 Negative |
| 4 | ANIMAL | 0.11 | >0.1 Positive |
| | DANDER | | |
| 5 | EGG WHITE | 0.03 | <0.1 Negative |
| 6 | CHEESE | 0.05 | >0.1 Positive |
| | CHEDDAR | | |
| 7 | GREEN PEA | 0.06 | <0.1 Negative |
| 8 | COCONUT | 0.04 | >0.1 Positive |
| 9 | GARLIC | 0.02 | <0.1 Negative |
| 10 | GLUTEN | 0.05 | >0.1 Positive |
| 11 | EGG YOLK | 0.01 | <0.1 Negative |
| 12 | PEANUT | 0.10 | >0.1 Positive |
| 13 | CABBAGE | 0.03 | <0.1 Negative |
| 14 | CASHEW | 0.02 | >0.1 Positive |
| 15 | BANANA | 0.05 | <0.1 Negative |
| 16 | CORN | 0.04 | >0.1 Positive |
| 17 | CAULIFLOWER | 0.03 | <0.1 Negative |
| 18 | POTATO | 0.02 | >0.1 Positive |
| 19 | SILK | 0.09 | <0.1 Negative |
| 20 | COFFEE | 0.02 | >0.1 Positive |
| 21 | LEMON | 0.09 | <0.1 Negative |
| 22 | MUSTARD | 0.03 | >0.1 Positive |
| 23 | APPLE | 0.06 | <0.1 Negative |
| 24 | ONION | 0.01 | >0.1 Positive |
| 25 | TOMATO | 0.05 | <0.1 Negative |
| 26 | CARROT | 0.06 | >0.1 Positive |
| 27 | PLUM | 0.05 | <0.1 Negative |
| 28 | VELVET | 0.04 | >0.1 Positive |
| 29 | LENTIL | 0.02 | <0.1 Negative |
| 30 | DUST | H 0.17 | >0.1 Positive |
| 31 | POLLEN | H 0.23 | >0.1 Positive |
| | Total IgE | 184 | <1.5 −378 IU/ml |

His allergy profile indicated that he was allergic to pollen and dust allergens. As per the aforementioned allergy profile a suitable formulation was designed. The formulation contained a mixture of potentized dispersions of pollen and dust allergens having potency of 100c and 150c respectively. The dose of the formulation was 0.2 ml 3 times a day for 2 weeks.

After 15 days his allergy profile was again identified.

| Sr. | | IgE profile | Reference Range |
|---|---|---|---|
| No. | Allergen | (kUA/L) | (kUA/L) |
| 1 | MILK | 0.06 | <0.1 Negative |
| 2 | RICE | 0.10 | >0.1 Positive |
| 3 | WHEAT | 0.10 | <0.1 Negative |
| 4 | ANIMAL | 0.04 | >0.1 Positive |
| | DANDER | | |
| 5 | EGG WHITE | 0.03 | <0.1 Negative |
| 6 | CHEESE | 0.03 | >0.1 Positive |
| | CHEDDAR | | |
| 7 | GREEN PEA | 0.06 | <0.1 Negative |
| 8 | COCONUT | 0.04 | >0.1 Positive |
| 9 | GARLIC | 0.02 | <0.1 Negative |
| 10 | GLUTEN | 0.06 | >0.1 Positive |
| 11 | EGG YOLK | 0.01 | <0.1 Negative |
| 12 | PEANUT | 0.10 | >0.1 Positive |
| 13 | CABBAGE | 0.02 | <0.1 Negative |
| 14 | CASHEW | 0.03 | >0.1 Positive |
| 15 | BANANA | 0.05 | <0.1 Negative |
| 16 | CORN | 0.04 | >0.1 Positive |
| 17 | CAULIFLOWER | 0.06 | <0.1 Negative |
| 18 | POTATO | 0.02 | >0.1 Positive |
| 19 | SILK | 0.09 | <0.1 Negative |
| 20 | COFFEE | 0.02 | >0.1 Positive |
| 21 | LEMON | 0.05 | <0.1 Negative |
| 22 | MUSTARD | 0.03 | >0.1 Positive |
| 23 | APPLE | 0.06 | <0.1 Negative |
| 24 | ONION | 0.01 | >0.1 Positive |
| 25 | TOMATO | 0.05 | <0.1 Negative |
| 26 | CARROT | 0.08 | >0.1 Positive |
| 27 | PLUM | 0.05 | <0.1 Negative |
| 28 | VELVET | 0.02 | >0.1 Positive |
| 29 | LENTIL | 0.02 | <0.1 Negative |
| 30 | DUST | H 0.15 | >0.1 Positive |
| 31 | POLLEN | H 0.20 | >0.1 Positive |
| | Total IgE | 170 | <1.5 −378 IU/ml |

The aforementioned allergy profile indicated that, IgE level for the allergens such as pollen and dust has been reduced indicating effectiveness of the present formulation. It was also observed that IgE level for pollen and dust allergens still need to be reduced.

Accordingly, a suitable formulation was designed. The formulation contained a mixture of potentized dispersions of pollen and dust allergens having potency of 80c and 50c. The dose of the formulation was prescribed as 0.2 ml 3 times a day for 2 weeks.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

“Whenever a range of values is specified, a value up to 10% below and above the lowest and highest numerical value respectively, of the specified range, is included in the scope of the invention”.

While considerable emphasis has been placed herein on the particular features of this invention, it will be appreciated that various modifications can be made, and that many changes can be made in the preferred embodiments without departing from the principle of the invention. These and other modifications in the nature of the invention or the preferred embodiments will be apparent to those skilled in the art from the disclosure herein, whereby it is to be distinctly understood that the foregoing descriptive matter is to be interpreted merely as illustrative of the invention and not as a limitation.

1. A method for the treatment of allergic conditions; said method comprising the following steps:
a. obtaining the allergy profile of an individual, identifying allergens to which the individual is susceptible and the level of susceptibility;
b. arranging the identified allergen according to the sensitivity;
c. diluting 1 μg to 1 mg mass of each of the identified allergen individually with at least one vehicle to obtain a primary dilution of 1c potency;
d. serially diluting with potentization, each of the primary dilutions with the vehicle in a ratio of 1:9 to 50:50 to obtain potentized dispersions for each of the identified allergens; characterized in that the potencies of said potentized dispersions are in the range of 2c to 100000c; preferably in the range of 30c to 500c;
e. mixing the potentized dispersions of each of the identified allergens to obtain a formulation; characterized in that the potency of each of said dispersions is directly proportional to the sensitivity of the individual to the identified allergen;
f. administering a predetermined amount of the formulation having a predetermined potency to the individual for a predetermined period;
g. retesting the allergy profile of the individual after a predetermined period; and
h. repeating at least once the method steps of (a) to (f) after readjusting the potencies of the individually identified allergens depending upon the retested allergy profile, until a significant reduction in said allergic conditions is observed.

2. The method as claimed in claim 1, wherein the allergen is at least one selected from the group consisting of pollen, animal dander, fungal spores, dust, venoms, food, biochemical allergens and chemical allergens.

3. The method of as claimed claim 1, wherein the vehicle is at least one selected from the group consisting of normal saline, distilled water and ethyl alcohol.

4. The method of as claimed claim 1, wherein the formulation is in a wet dosage form.

5. The method of as claimed claim 1, wherein the amount of the formulation administered per day is in the range of 0.01 ml to 1 ml.

6. A formulation comprising potentized dispersions of each of the identified allergens to which an individual is susceptible; characterized in that the potency of said potentized dispersion is directly proportional to the sensitivity of the individual to the identified allergen.

7. The formulation as claimed in claim 6, wherein the allergen is at least one selected from the group consisting of pollen, animal dander, fungal spores, dust, venoms, food, biochemical allergens and chemical allergens.

8. The formulation as claimed in claim 6, wherein the potency of the potentized dispersion is in the range of 2c to 100000c; preferably, in the range of 30c to 500c.

9. The formulation as claimed in claim 6, wherein the potentized dispersion comprises a vehicle and the allergens to which an individual is susceptible; wherein the vehicle is at least one selected from the group consisting of normal saline, distilled water and ethyl alcohol.

10. The formulation as claimed in claim 6, wherein the formulation is in a wet dosage form.

11. A process for preparing a formulation; said process comprising:
a. obtaining allergens to which an individual is susceptible;
b. diluting 1 μg to 1 mg mass of each of the identified allergen individually with at least one vehicle to obtain a primary dilution of 1c potency;
c. serially diluting with potentization, each of the primary dilutions with the vehicle in a ratio of 1:9 to 50:50 to obtain potentized dispersions for each of the identified allergens; characterized in that the potencies of said potentized dispersions are in the range of 2c to 100000c; preferably, in the range of 30c to 500c; and
d. mixing the potentized dispersions to obtain a formulation; characterized in that the potency of said dispersion is directly proportional to the sensitivity of the individual to the identified allergens.

12. The process as claimed in claim 11, wherein the allergen is at least one selected from the group consisting of pollen, animal dander, fungal spores, dust, venoms, food, biochemical allergens and chemical allergens.

13. The process as claimed in claim 11, wherein the vehicle is at least one selected from the group consisting of normal saline, distilled water and ethyl alcohol.

14. The process as claimed in claim 11, wherein the formulation is in a wet dosage form.

15. The process as claimed in claim 11, wherein the method step of potentization is carried out by exerting a force of at least 6 dynes rhythmically at a frequency of 10 strokes in two minutes.

16. A kit comprising (a) at least one allergen, (b) at least one vehicle and (c) accessories which include bottles and mixing sticks.

17. The kit as claimed in claim 16, wherein the allergen is at least one selected from the group consisting of pollen, animal dander, fungal spores, dust, venoms, food, biochemical allergens and chemical allergens.

18. The kit as claimed in claim 16, wherein the vehicle is at least one selected from the group consisting of normal saline, distilled water and ethyl alcohol.
